(19) United States
(12) Patent Application Publication
(10) Pub. No.: US 2026/0350519 A1
(43) Pub. Date: Nov. 5, 2026

(54) EXPLAINABLE ARTIFICIAL INTELLIGENCE-BASED SALES MAXIMIZATION DECISION MODELS

(71) Applicant: PharmaForceIQ, Inc., Miami, FL (US)

(72) Inventors: Marc Cohen, San Francisco, CA (US); Pinchas Ben-Or, San Francisco, CA (US)

(21) Appl. No.: 19/391,703

(22) Filed: Nov. 17, 2025

Related U.S. Application Data
(63) Continuation of application No. 18/051,258, filed on Oct. 31, 2022, now abandoned.
Application No. 18/051,258 is a continuation of application No. 17/110,157, filed on Dec. 2, 2020, now abandoned.
Application No. 17/110,157 is a continuation of application No. PCT/US2020/035773, filed on Jun. 2, 2020.
(60) Provisional application No. 62/934,955, filed on Nov. 13, 2019.
(60) Provisional application No. 62/948,719, filed on Dec. 16, 2019.

(51) Int. Cl. G06Q 10/0637; G06N 5/04; G06N 20/00; G06Q 10/04; G06Q 10/10; G06Q 10/107; G06Q 30/0202; G16H 40/20; G16H 50/70; G16H 70/40

(52) U.S. Cl. CPC G06Q 10/06375; G06N 5/04; G06N 20/00; G06Q 10/04; G06Q 10/103; G06Q 10/107; G06Q 30/0202; G16H 40/20; G16H 50/70; G16H 70/40

(57) ABSTRACT

The present disclosure provides systems, methods, and computer program products for explaining decision models. An example method may comprise (a) generating one or more predictive models; (b) generating a decision model from the one or more predictive models by imposing (i) a set of operational constraints and (ii) a set of brand strategy rules on the predictive model; (c) using the decision model to determine one or more optimal actions for maximizing one or more target variables; and (d) applying explainability modeling to the decision model to generate an explanation model, wherein the explanation model is useable by one or more users to gain insights or an understanding into interactions within the decision model affecting the sales of the one or more products.

### CROSS-REFERENCE

This application is a continuation application of U.S. application Ser. No. 18/051,258, filed on Oct. 31, 2022, which is a continuation application of U.S. application Ser. No. 17/110,157, filed on Dec. 2, 2020, which is a continuation application of International Application No. PCT/US2020/035773, filed on Jun. 2, 2020, which claims priority to U.S. Provisional Patent Application No. 62/934,955, filed on Nov. 13, 2019, and U.S. Provisional Patent Application No. 62/948,719, filed on Dec. 16, 2019, each of which is incorporated herein by reference in its entirety.

### BACKGROUND

Machine learning (ML) models are algorithms that can be trained to predict or classify one or more outputs from one or more inputs. ML models can classify data, predict features of data, and make recommendations based on data. However, ML models may be very complex; they may receive thousands of features as input and have thousands of parameters, and the parameters may be non-linear. Additionally, the underlying structure and function of an ML model may be opaque. In other words, it may be unclear to a human user how the ML model interprets certain data and why the ML model generates particular outputs. Practical AI technology typically includes additional elements beyond ML models, such as decision models involving rules and optimization. Explainable artificial intelligence (xAI) is an area of research dedicated to developing approaches to explain how and why ML and AI models generate the outputs that they do.

### SUMMARY

The present disclosure provides methods for explaining models that drive decision-making processes for businesses and involves extending xAI beyond ML models construed narrowly to the more general category of AI decision models. Such models may be referred to as “decision models” in this disclosure. A decision model may contain a predictive model, or it may be based in some way on a predictive or classifying ML model that is trained on historical data and for many practical applications may be limited by one or more constraints. The constraints may be operational constraints imposed on the business that limit the range of practical outputs that the predictive model can generate. Additionally or alternatively, the constraints may be rules set by the business that align with the goals of the business that likewise limit the range of outputs that the decision model can generate. The trained decision model can determine one or more optimal actions for maximizing one or more target variables. The target variables may be business metrics, e.g., sales metrics. The methods described herein can comprise generating an explanation model from the decision model. The explanation model may be useable to gain insight into the structure and function of the model.

The methods described above can enable an organization to better understand the decision models that it uses and persuade stakeholders within the organization to trust such models and follow their decisions. This may be particularly desirable in the field of pharmaceutical sales, in which the use of decision models to drive physician interactions has increased substantially. Such decision models have evolved to manage decisions on how, when, and what to say to physicians to improve pharmaceutical sales and physician engagement. To be most effective, such decision models may integrate brand strategy, business constraints, and models that are predictive of human behavior. While the effect of each of these factors may be individually understandable, the behavior of the composite decision model may be much harder to explain. This may be particularly true for decision models that rely on ML-based analytics. Even if the decision model is not business rule-constrained and solely relies on a single ML or artificial intelligence (AI) model, its decisions may need to be understandable to be persuasive to stakeholders. For example, if a decision model recommends that a sales representative deliver a particular message to a physician in-person, it may be important for the sales representative to know why the system made such a recommendation so that the representative gains confidence in the recommendation (and in the system more generally) and follow the recommendation.

In an aspect, the present disclosure provides a computer-implemented method for enhancing explainability of one or more models that are useable to increase sales of one or more products. The method may comprise: generating one or more predictive models based at least in part on (i) a set of target variables, (ii) a set of features, and (iii) a set of decision variables, wherein the features are predictive of and have an influence on the target variable, and wherein the decision variables are a subset of the set of features; generating a decision model by imposing (i) a set of operational constraints and (ii) a set of brand strategy rules on the one or more predictive models, wherein the set of operational constraints comprises logistical constraints associated with one or more sales representatives that interact with one or more target personnel to promote a use of the one or more products, and wherein the set of brand strategy rules is defined by one or more entities that are offering the one or more products for sale; using the decision model to determine one or more optimal actions for maximizing one or more target variables within the set of target variables; and applying explainability modeling to the decision model and the one or more optimal actions to generate an explanation model, wherein the explanation model is useable by one or more users to gain insights or an understanding into interactions within the decision model affecting the sales of the one or more products.

In some embodiments, the one or more target personnel may comprise a health care provider (HCP). The one or more products may comprise a pharmaceutical product. The target variables may comprise one or more categorical and/or continuous variables associated with one or more actions taken by the HCP.

Decision models may also be implemented outside of the healthcare and pharmaceutical sectors. For example, decision models may be implemented the in retail, financial services, and consumer products sectors. Decision models may also be used with military, transport, and robotics technologies. For example, decision models may be used to provide insight into predictions made by complex financial models, or help military officials extract insights from intelligence reports or sensor data. Additionally, decision models may help to explain factors that drive consumers to retail stores and away from online shopping.

In some embodiments, the one or more actions in the above method may comprise: (1) the HCP opening an email correspondence that is sent to the target by the one or more sales representatives, or (2) the HCP reading an online report associated with the pharmaceutical product.

In some embodiments, the target variables may comprise one or more continuous variables associated with the pharmaceutical product, wherein the one or more continuous variables comprise a prescription, market share, or sales for the pharmaceutical product.

In some embodiments, the set of features may comprise demographic data associated with the HCP. The demographic data may comprise age, gender, educational background, and segment membership of the HCP. The set of features may comprise patient data indicative of the HCP's patient population characteristics. The set of features may comprise contact history associated with communications between the HCP and the one or more sales representatives. In some embodiments, the contact history may comprise one or more of the following: (1) a number of visits by the one or more sales representatives to the HCP, (2) topics of conversations during the visits, (3) a number of email correspondences sent by the one or more sales representatives to the HCP, (4) topics of the email correspondences sent, (5) documents relating to the pharmaceutical product provided by the one or more sales representatives to the HCP, (6) webinars attended by the one or more sales representatives and the HCP, and (7) conferences attended by the one or more sales representatives and the HCP.

In some embodiments, the set of decision variables may comprise actions and timings that are controllable and executed by the one or more sales representatives or by a third-party.

In some embodiments, the logical constraints may be associated with one or more of the following: (1) maintaining a pacing of visits by the one or more sales representatives to the HCP, (2) coordinating the visits with non-face-to-face interactions, or (3) the one or more sales representatives traversing a territory in a systematic or efficient manner.

In some embodiments, the one or more entities that define the set of brand strategy rules may comprise brand management and sales operations teams for the pharmaceutical product.

In some embodiments, the set of target variables may comprise a sales deviation from a mean group facility sales. In some embodiments, the one or more predictive models may be built using random forest regression with a selected target being the sales deviation from the mean group facility sales.

In some embodiments, the explanation model may be generated by using a set of counterfactuals to generate a plurality of observations that cover a space of a plurality of predictors. The plurality of predictors may comprise one or more of the following: (1) a medical facility having a number of HCPs, (2) a number of unscheduled visits to the HCPs within the medical facility, or (3) a fiscal quarter in which sales data is collected.

In some embodiments, applying the explainability modeling may comprise using recursive partitioning over the entire space to enable insight into covariate relationships.

In some embodiments, the explanation model may comprise a global explanation model. Alternatively, the global explanation model may comprise an unconstrained global decision tree. In some embodiments, the global explanation model may comprise a constrained global decision tree.

In some embodiments, applying the explainability modeling may comprise using recursive partitioning to a margin of the space instead of over the entire space.

In some embodiments, the explanation model may comprise a local explanation model. The local explanation model may comprise a local decision tree.

In some embodiments, the explanation model may be useable by the one or more users to make optimal decisions in a domain of marketing analytics, one-to-one marketing, and personalization of recommendations to increase the sales of the one or more products.

Another aspect provides a system for enhancing explainability of one or more models that are useable to increase sales of one or more products. The system may comprise: one or more processors; and a memory storing instructions that, when executed by the one or more processors, cause the one or more processors to perform operations comprising: generating one or more predictive models based at least in part on (i) a set of target variables, (ii) a set of features, and (iii) a set of decision variables, wherein the features are predictive of and have an influence on the target variables, and wherein the decision variables are a subset of the set of features; generating a decision model by imposing (i) a set of operational constraints and (ii) a set of brand strategy rules on the one or more predictive models, wherein the set of operational constraints comprises logistical constraints associated with one or more sales representatives that interact with one or more target personnel to promote the use of the one or more products, and wherein the set of brand strategy rules is defined by one or more entities that are offering the one or more products for sale; using the decision model to determine one or more optimal actions for maximizing one or more target variables within the set of target variables; and applying explainability modeling to the decision model and the one or more optimal actions to generate an explanation model, wherein the explanation model is useable by one or more users to gain insights or an understanding into interactions within the decision model affecting the sales of the one or more products.

A further aspect provides a non-transitory computer-readable storage medium including instructions that, when executed by one or more processors, cause the one or more processors to perform operations comprising: generating one or more predictive models based at least in part on (i) a set of target variables, (ii) a set of features, and (iii) a set of decision variables, wherein the features are predictive of and have an influence on the target variables, and wherein the decision variables are a subset of the set of features; generating a decision model by imposing (i) a set of operational constraints and (ii) a set of brand strategy rules on the one or more predictive models, wherein the set of operational constraints comprises logistical constraints associated with one or more sales representatives that interact with one or more target personnel to promote the use of the one or more products, and wherein the set of brand strategy rules is defined by one or more entities that are offering the one or more products for sale; using the decision model to determine one or more optimal actions for maximizing one or more target variables within the set of target variables; and applying explainability modeling to the decision model and the one or more optimal actions to generate an explanation model, wherein the explanation model is useable by one or more users to gain insights or an understanding into interactions within the decision model affecting the sales of the one or more products.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a diagram of various modeling techniques;

FIG. 2 schematically illustrates a system that can generate a decision model and an explainability model of the decision model;

FIG. 3 is a flow chart of an example process for generating an explanation model of a decision model;

FIG. 4 shows distributions of data for training a predictive model; values;

FIG. 5 shows scatter plots of a predictive model's predicted values against target

FIG. 6A and FIG. 6B show a surface of predictions of a predictive model;

FIG. 7A and FIG. 7B show plots that track a target variable of a predictive model against several combinations of predictors;

FIG. 8 shows global explanation trees of a decision model;

FIG. 9A and FIG. 9B show local explanation trees of a decision model;

FIGS. 10A, 10B, and 10C show LIME coefficients of a decision model; and

FIG. 11 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The present disclosure provides methods for explaining models that drive decision-making processes for businesses. Such models may be referred to as “decision models” in this disclosure. A decision model may include a predictive model, e.g., a machine learning (ML) model, that is trained on historical data and limited by one or more constraints and identifies decisions that optimize some business financial objective. The constraints may be operational constraints imposed on the business that limit the range of practical outputs that the predictive model can generate. Additionally or alternatively, the constraints may be rules set by the business that align with the goals of the business that likewise limit the range of decision outputs that the predictive model can generate and which optimizes the business objective. The trained decision model can determine one or more optimal actions for maximizing one or more target variables. The target variables may be business metrics, e.g., sales metrics. The methods described herein can comprise generating an explanation model from the decision model. The explanation model may be useable to gain insight into the structure and function of the model.

Before the popularization of ML and artificial intelligence (AI) models, statistical models were generally designed to be predictive and interpretable. A model may be “interpretable” if a person can understand the impact of a predictor or group of predictors on the target variable that the model determines. Alternatively or additionally, a model may be “interpretable” if (i) a person can understand the model enough to make accurate predictions about its behavior on untested data or (ii) if a person has enough confidence in the model to believe in it. Such interpretable models were designed to distinguish between the effects of particular predictors on the target variable with a high degree of certainty. To that end, interpretable models were typically parametric and often linear. The parameters of such parametric models were designed to provide insight into the underlying relationship between the predictors and the target variable.

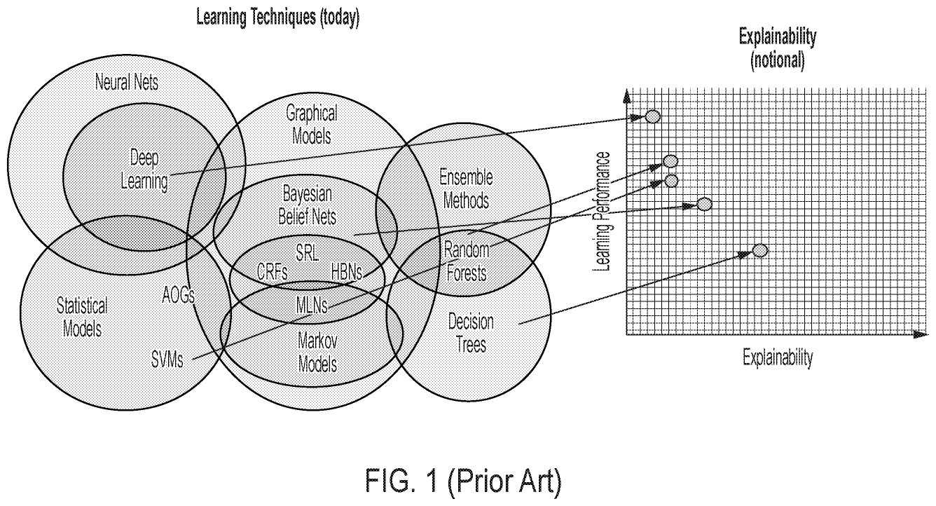

State-of-the-art models today are generally more complex and more opaque than traditional parametric models. Such models include deep neural networks and ensemble models. Understanding the role that predictors play in complex ML models is currently referred to as “explainable AI” (xAI) or “explainability.” FIG. 1 is a diagram of various modeling techniques from Gunning, D. “Explainable Artificial Intelligence (XAI),” which is incorporated by reference herein in its entirety.

### Explainability Models

Explainability models may be models that are inherently interpretable or models that explain other uninterpretable models. Explainability models may include deep explanation models, interpretable models, and models of models (“model induction”). Deep explanation models are neural networks in which nodes are identified as features so that the weights of the various layers illuminate the drivers of the neural network. Interpretable models are models that are inherently interpretable, including linear models, parametric models, tree models, Bayesian models, and the like. And model induction is a technique whereby a more interpretable model is built on top of an underlying model. Examples of models that may be used in model induction are local interpretable model-agnostic explanations (LIME), Shapley additive explanations (SHAP), counterfactual local explanations via regression (CLEAR), Anchors, and leave one covariate out (LOCO).

Explainability models may be local or global. Local explainability models may explain a specific prediction of the underlying model, namely at a single point in the space of training or test data. In the context of image classification, for example, a local explainability model may identify the drivers that result in a particular image being classified in a particular way. In general, local explainability models may provide explanations that describe the local behavior of the model using a linearly weighted combination of the input features. Linear functions can capture relative importance of features in an easy-to-understand manner. Global explainability models, meanwhile, may seek to explain a large range of unseen instances.

### Local Explainability Models

One example of a local explainability model is LIME. LIME is a technique that fits a linear model to a particular data sample (e.g., a set of input features). The linear model may have coefficients that each indicate the amount that a particular feature contributes to the output of the underlying model. LIME may determine the coefficients by perturbing the input features and observing the resulting impact on the output of the underlying model. LIME may save a collection of weighted predictions of the underlying model at sampled instances around the data sample. The weights may be based on the distance to the data sample. The linear approximation of the model may be used to explain the behavior of the more complex underlying model.

Another example of a local explainability model is Anchors. Anchors, unlike LIME, may account for interaction effects and may more accurately attribute explanations in text mining applications. Anchors looks for a set of features such that if any features not in that set are included the predictions do not change “substantively.” “Substantively” is defined by the expected value of the likelihood of a change in prediction being less than a prescribed amount. Anchors may be computationally complex since a large space may need to be searched in order to satisfy the Anchors criteria.

Another example of a local explainability model is CLEAR. CLEAR exploits the use of counterfactuals and also expands on the univariate limitations of LIME. CLEAR uses the concept of w-counterfactuals to explain a prediction by answering the question of “what if things had been different” with the feature set. Rather than randomly sampling the data and weighing such data by proximity to the point of interest as in LIME, the CLEAR method to is systematically search the space around the data point of interest and evaluate the model at those points producing counterfactuals to identify classification changes. The points at which this occurs can then be used to build a regression model for explanation thus improving the fidelity of the explanation around the point in question.

Another example of a local explainability model is LOCO. LOCO may generate metrics that measure variable importance. The metrics may be based on differences in errors from a complete model or a model built without one of the covariates. A metric can be analyzed in a local manner or a global manner by applying it to every instance in the test data set and then analyzing the distribution of the variable importance metric. The single instance metric is similar to the variable importance measure used in random forests by analyzing the decrease in node purity by changing the order of variable splits.

### Global Explainability Models

One example of a global explanation model is Shapley Additive Explanations (SHAP). SHAP is a unified framework for interpreting predictions; it assigns each feature an importance value of a particular prediction. In this way it is similar to some of the local approaches described above.

One framework for SHAP is additive feature attribution methods, which provides a representation of relative feature importances within a prediction model. Additive feature attribution may estimate an underlying prediction model as a sum of transformed, weighted feature terms. The method may determine the weights by minimizing a loss function. Features which are more heavily weighted may be thus inferred to be more important to the prediction.

This is similar to LOCO in that a new model is built for each predictor leaving the predictor out and then that new model is evaluated at the point of interest and the difference in the value of the prediction with the prediction from the full model is weighted by the non-zero occurrences for that predictor. Other global explainability models include partial dependence plots, recursive partitioning, decision tree methods, and the like.

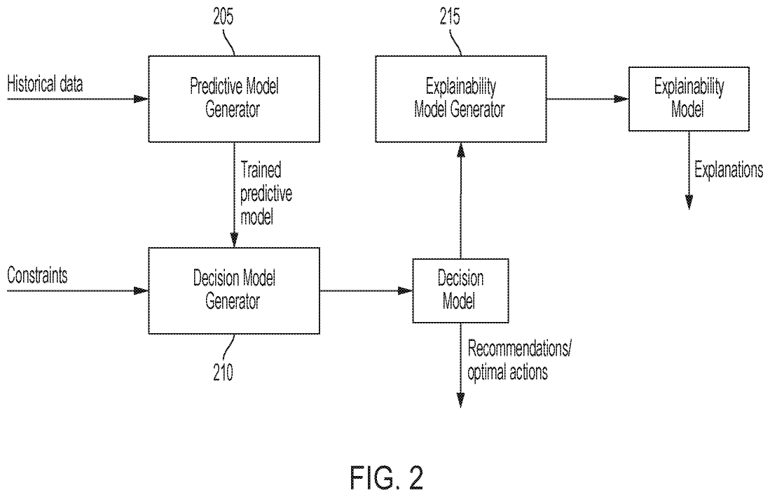

FIG. 2 schematically illustrates a system 200 that can generate a decision model and an explainability model of the decision model. The decision model may be a model that makes recommendations to a person or entity (e.g., a business). The recommendations may be actions that minimize, maximize, or otherwise optimize target variables of interest to the person or entity. For example, a decision model for a sales organization may recommend that a sales representative initiate a customer contact that maximizes the likelihood that the customer purchases a product. The recommendation may include the substance, time, and mode (e.g., in-person, telephone call, or email) of the customer contact.

The decision model may be so complex that its behavior is opaque and requires explanation. The system 200 can generate an explainability model of the decision model that, for each recommendation, generates an explanation that demonstrates why the decision model made the particular recommendation that it did. For example, with continued reference to the decision model for the sales organization described above, the explainability model can generate an explanation that demonstrates why the decision model recommended a particular mode of customer contact.

The system 200 can include a predictive model generator 205. The predictive model generator 205 can generate a predictive model \hat{f}(X)=Y. Y may be a target variable. Y may be a categorical target variable, such as whether a customer will take a particular action (e.g., open an email, answer a phone call, read an online report, purchase an offered product, etc.). Alternatively, Y may be a continuous target variable, such as the market share for a product that a sales organization offers or the perception of the sales organization by customers.

X may be features that are predictive or believed to be predictive of the target variable Y. With continued reference to a predictive model for a sales organization, X may include demographic information of about a customer (e.g., age, gender, educational background, and the like). The demographic profile of a customer may, for example, be predictive of the type of communication that the customer prefers to receive (e.g., a phone call rather than an email). X may also include data about the customer's business. For example, if the sales organization is a pharmaceutical sales organization and the customer is a health care provider (“HCP”), X may include data about the HCP's patient population. X may also include a history of previous contacts with the customer, including the substance, dates and times, and outcomes of in-person visits to the customer, emails sent to the customer, documents provided to the customer, webinars and conferences attended by the customer, and the like. X may be configured in multiple ways, depending on whether the prediction model is time-dependent or not.

The predictive model generator 205 can use historical data, including historical values for X and Y, to find (e.g., train) a model f(X)=Y that can be used to predict future Y values. Regardless of the training method, the trained model may not be perfect. Therefore, the trained model can be represented as \hat{f}( )=Y such that the error associated with the model is Y−\hat{f}(X). A successful decision model may explain to humans why \hat{f}( ) is predictive by identifying some set of variables within X as decision variables. These decision variables may be variables on which humans may have control, and thus may allow humans to calibrate or optimize their actions (e.g., contacts from pharma reps to HCPs) to achieve desired results (e.g., increased sales or prescriptions filled). The values of decision variables that achieve desired results may not be feasible in the real world. Further, entities (e.g., businesses or regulators) may bar persons from taking actions represented by decision variables. In these situations, the system may add constraints to the decision model to better simulate real-world conditions or reflect real-world needs.

The predictive model generator 205 can train the predictive model using a supervised, semi-supervised, or unsupervised learning process, for example. A supervised predictive model can be trained using labeled training inputs, i.e., features X and corresponding target variables Y. Features X can be provided to an untrained or partially trained version of the predictive model to generate a predicted output. The predicted output can be compared to the known target variable Y for that set of features X, and if there is a difference, the parameters of the predictive model can be updated. A semi-supervised predictive model can be trained using a large number of unlabeled features X and a small number of labeled features X. An unsupervised predictive model, e.g., a clustering or dimensionality reduction model, can find previously unknown patterns in features X.

The predictive model generated by the predictive model generator 205 may be a neural network (e.g., a feedforward neural network, a convolutional neural network (CNN), a recurrent neural network (RNN), a long short-term memory network (LSTM), etc.), an autoencoder, a regression model, a decision tree, a random forest model, a support vector machines, a Bayesian network, a clustering model, a reinforcement learning algorithm, or the like.

The system 200 can also include a decision model generator 210. The decision model generator 210 can generate a decision model from the predictive model. The decision model can predict the values of decision variables D that maximize the target variable Y, where decision variables D are a subset of features X. Decision variables may be variables over which a person or entity has some control. For example, a sales representative can control the content and timing of emails, topics of discussion on a phone call, and the like. The predictive problem may therefore be recharacterized as f(X,D)=Y. The goal of finding f( ) may be to use the information contained therein to make decisions about what values of D maximize Y. This may be expressed as the unconstrained decision model:

$$d * ( x ) ≡ argm \frac{a}{d} x \overline{f ( x , d )} .$$

In practice, all possible choices for d( ) may not be feasible from a business perspective. A such, the decision model generator 210 may take into account certain constraints when generating the decision model from the predictive model. For example, maximizing the likelihood that a customer purchases a product may require visiting the customer immediately. While that may be desirable, it may not be feasible because of logistical realities (e.g., a sales representative or the customer may not be available immediately). Other examples of constraints for sales organizations may be maintaining a pacing of visits, coordinating visits with non-face-to-face interactions, traversing the territory systematically. These constraints may be denoted by C. Therefore, d*(x) may be denoted by

$$d * ( x ) ≡ argm \frac{a}{d ∈ c} x \overline{f ( x , d )} ,$$

where dεC denotes that the searchable space of d values that satisfies the constraints.

In practice, brand management and sales operations teams may also specify certain rules. Such rules may result from various plans and goals that may not be captured in the relationship between (X,D) and Y. For example, a brand team may want to prioritize the sale of a new product on the marketplace. Additionally or alternatively, the brand team may specify rules for interacting with uncontrolled publications, rules that require visits when commercial metrics change in statistically relevant ways, rules for timing interactions with seasonal commercial drivers, rules for coordinating messaging across products brands, and the like. Let R denote the set of rules and D denote the union of constraints and rules, namely D=C U R. The constrained decision model may therefore be denoted by

$$d * ( x ) ≡ argm \frac{a}{d ∈ D} x \overline{f ( x , d )} .$$

The constrained decision model can generate recommendations that are predicted to maximize the target variable Y. Although d*(x) is as presented is based on a single fitted model, in practice, the function being optimized could be an algorithm with many components including heuristics, raw data, feature engineered data, and the results of statistical and machine-learned models. This generality does not change the explainability approach presented below.

The system 200 can also include an explainability model generator 215. The explainability model generator 215 can generate an explainability model of the decision model. The explainability model may generate a local or global explanation of the decision model, which may be desirable if the decision model is opaque or otherwise difficult to understand.

Explaining a decision model may be more complex than explaining a traditional classification model. A classification model determines whether an instance is in a target group or not. Decision models may be more complex in that the output may not be a binary or even multi-class classification, but an optimization based on one or more decision variables. However, the need to understand what is driving the optimization is just as important. In many practical cases, a person or entity may be reluctant to rely on an opaque model that simply outputs a decision. The person or entity may require a deeper understanding of the structure and function of the model and what areas of the predictor space lead to specific decisions.

The explainability models previously described herein can be applied to understanding and explaining decision models. Explainability models of decision models will be described in more detail with reference to an example below.

The subsystems of FIG. 2 and their components can be implemented on one or more computing devices. The computing devices can be servers, desktop or laptop computers, electronic tablets, mobile devices, or the like. The computing devices can be located in one or more locations. The computing devices can have general-purpose processors, graphics processing units (GPU), application-specific integrated circuits (ASIC), field-programmable gate-arrays (FPGA), or the like. The computing devices can additionally have memory, e.g., dynamic or static random-access memory, read-only memory, flash memory, hard drives, or the like. The memory can be configured to store instructions that, upon execution, cause the computing devices to implement the functionality of the subsystems. The computing devices can additionally have network communication devices. The network communication devices can enable the computing devices to communicate with each other and with any number of user devices, over a network. The network can be a wired or wireless network. For example, the network can be a fiber optic network, Ethernet® network, a satellite network, a cellular network, a Wi-Fi® network, a Bluetooth® network, or the like. In other implementations, the computing devices can be several distributed computing devices that are accessible through the Internet. Such computing devices may be considered cloud computing devices.

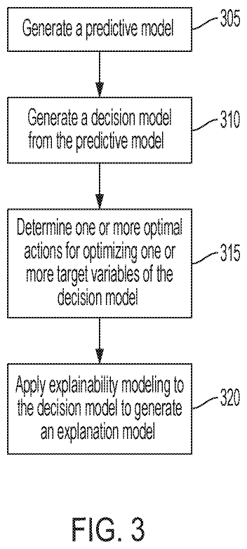

FIG. 3 is a flow chart of an example process 300 for generating an explanation model of a decision model. The process 300 can be performed by the system 200 of FIG. 2, which may be implemented on one or more appropriately-programmed computers in one or more locations.

The system can generate a predictive model (305). The predictive model may be configured (e.g., trained) to determine a target variable from a set of features. In general, the predictive model may be a model that is opaque or otherwise a “black box.” That is, the structure and function of the predictive model may not be easily interpretable by a user. The predictive model may be a ML or AI model. The ML or AI model may be a neural network (e.g., a feedforward neural network, a convolutional neural network (CNN), a recurrent neural network (RNN), a long short-term memory network (LSTM), etc.), an autoencoder, a regression model, a decision tree, a random forest model, a support vector machine, a Bayesian network, a clustering model, a reinforcement learning algorithm, or the like.

The target variable may be a metric that a person or business is interested in minimizing, maximizing, or otherwise optimizing (e.g., revenue, profit, quantity of customers or users, production time, shipping time, customer rating, customer response rate, etc.) The target variable may be a categorical variable. That is, the target variable may be limited to a discrete number of values. For example, the target variable may be a determination that a particular event will or will not occur or that a particular action will or will not be taken. A pharmaceutical company, for example, may be interested in whether a health care provider (HCP) will take a particular action in response to a contact from a sales representative (e.g., open an email correspondence that is sent to the HCP by the sales representative, or read an online report associated with a pharmaceutical product). Alternatively or additionally, the target variable may be a continuous variable. That is, the target variable may take a number of values within a continuous range. A pharmaceutical company may be interested in the prescription, market share, or sales of a pharmaceutical product, for example.

In a particular example, the target variable may be the deviation in sales of a pharmaceutical product to a facility from the mean sales to comparable facilities (e.g., facilities in the same decile of sales as the facility).

The set of features may include features that are or are believed to be predictive of the target variable. The set of features may include decision variables. Decision variables may be actions that are under the control of and executed by the person or entity that implements or uses the predictive model (e.g., a sales representative). In other words, decision variables may be variables that can be deliberately controlled. The set of features may also include variables that cannot be controlled directly that are also predictive of the target variable. For example, a company's pre-existing market share, which the company may not be able to control directly, may be predictive of sales.

In the case of a pharmaceutical company, the set of features may include demographic data associated with an HCP. The demographic data may be predictive, for example, of whether the HCP will respond to a particular mode of contact but not another (e.g., a phone call, but not an email). The demographic data may include age, gender, education background, and the segment membership of the HCP. Additionally or alternatively, the set of features may include data that is indicative of the HCP's patient population (e.g., the percentage of the HCP's patient population that has a particular disease). Additionally or alternatively, the set of features may include a contact history associated with the HCP and sales representatives of the pharmaceutical company. The contact history may include one or more of the following: (1) a number of visits by the one or more sales representatives to the HCP, (2) topics of conversations during the visits, (3) a number of email correspondences sent by the one or more sales representatives to the HCP, (4) topics of the email correspondences sent, (5) documents relating to the pharmaceutical product provided by the one or more sales representatives to the HCP, (6) webinars attended by the one or more sales representatives and the HCP, and (7) conferences attended by the one or more sales representatives and the HCP. Such contact history and corresponding sales data may indicate which types of contact are most valuable to the pharmaceutical company.

The system can generate a decision model by imposing (i) a set of operational constraints and (ii) a set of brand strategy rules on the predictive model (310). The set of operational constraints may be logistical constraints that limit the potential actions that the person or entity using the decision model may take. For example, in the case of a sales organization, the logistical constraints may be constraints associated with how sales representatives interact with targets (e.g., potential clients or customers) to promote products. In the specific case of a pharmaceutical company, the targets may be HCPs, and the products may be pharmaceutical products. The logistical constraints may be, for example, (1) the number of appointments and visits that a sales representative is able to attend each day given the time available to him and his location, (2) coordinating visits with non-face-to-face interactions, or (3) the sales representative's realistic geographic range.

The brand strategy rules, on the other hand, may be plans and goals implemented by a brand strategy or sales operations team. For example, a brand team may want to prioritize the sale of a new product on the marketplace. Additionally or alternatively, the brand team may specify rules for interacting with uncontrolled publications, rules that require visits when commercial metrics change in statistically relevant ways, rules for timing interactions with seasonal commercial drivers, rules for coordinating messaging across products brands, and the like. While these are not logistical constraints, they still limit the potential actions that may be performed by sales representatives.

The system can determine one or more optimal actions for minimizing, maximizing, or otherwise optimizing the one or more target variables within the set of target variables (315).

The system can apply explainability modeling to the decision model to generate an explanation model (320). The explanation model may be useable by one or more users to gain insight into interactions within the decision model affecting the target variable. In some cases, the system may apply explainability modeling by applying recursive partitioning to the decision model to enable insight into covariate relationships between the set of features used to train the decision model. Recursive partitioning is a statistical method for multivariable analysis. Recursive partitioning may create a decision tree that strives to correctly classify members of a population by splitting the population into sub-populations based on several dichotomous independent variables. Each sub-population may in turn be split an indefinite number of times until the splitting process terminates after a particular stopping criterion is reached. The resulting decision tree may more clearly show a user how the decision model is actually making decisions.

In some cases, the system can apply other types of explainability modeling to the decision model, including other techniques described herein such as LIME, CLEAR, LOCO, or the like.

In some cases, the system may apply the explainability modeling (e.g., recursive partitioning) over the entire set of features used to train the decision model, resulting in a global explanation model (e.g., global decision tree). The global explanation model may be a constrained global explanation model in that it considers constraints applied to the decision model, or it may be an unconstrained global explanation model. In other cases, however, the system may apply explainability modeling over only a subset of the features used to train the decision model, e.g., a margin of the space instead of the entire space, resulting in a local explanation model. In the case of recursive partitioning, for example, this may result in a local decision tree.

The explanation model may be useable by one or more users to make optimal decisions in a domain of marketing analytics, one-to-one marketing, and personalization of recommendations to increase the sales of the one or more products. The system can present the explanation model to the one or more users is a visualization on a graphical user interface of a computing device. For example, the system can present the decision trees described herein in the user interface.

In a retail example, the set of features may include demographic and purchasing history associated with a particular customer. The features may be predictive as to whether the customer at a particular store, when the customer may make a purchase, what types of items the customer may purchase, or other target variables. The decision variables in such a scenario may be features under which retail companies or individual retail employees have some control, such as distribution of coupons and employee interactions with the customer. Thus, the decision model may determine the relative importances of the decision variables to a target outcome, while an explanation model may provide insight into how the decision variable features interact with one another.

Analogously, in a military example, the set of features may include topographical information from visual sensors of a particular drone or unmanned aerial vehicle (UAV). The features may be indicative as to which visible objects or areas are important for intelligence gathering or reconnaissance, as well as information about the drone and the flight path of the drone. Decision variables thus may include user-determined flight trajectories of the drone and configurations of the cameras on the drone. An explanation model may provide insight into which user actions may improve detections of objects of interest.

In a financial example, the set of features may include indicators of changes in stock prices. Some decision variables in this scenario may relate to actions that companies may take in the near term to impact stock prices. An explanation model may provide insight as to relationships between these actions, in order for companies to take actions which, by themselves, may increase stock prices while being less individually burdensome to the companies.

### Example

A pharmaceutical company wants to determine the quantity of quarterly visits to each facility that the company serves (e.g., doctor's offices, clinics, and hospitals) that maximizes the sale of each of two therapeutic products. The company is motivated to reduce costly individual visits, potentially replacing them with group conferences or emails and freeing up resources so that more facilities can be served with the same resource overhead. However, in-person visits may result in more sales. The company builds a decision model that determines the number of visits to each facility that maximizes the sale of the two therapeutics, considering historical data. The decision model is based on a predictive model f(x,d) that maps features, including facility visits, to sales. D*(x) may represent the constrained decision model.

### Data

The company trained the predictive model on historical sales data of the two products to different medical facilities. The historical sales data included quarterly sales data for each facility for each of the two products. A particular data record contained an indication of the product (product), quarter (qtr), and facility of the data record; a code indicating the decile of the sales of the facility (facility); the number of scheduled visits sales representatives made to an HCP in the facility (appointment); the number of conferences that HCPs within the facility attended (conference); the number of group meetings that HCPs within the facility attended (group); the number of emails sent to HCPs within the facility (email); and the number of unscheduled visits to HCPs within the facility (visit).

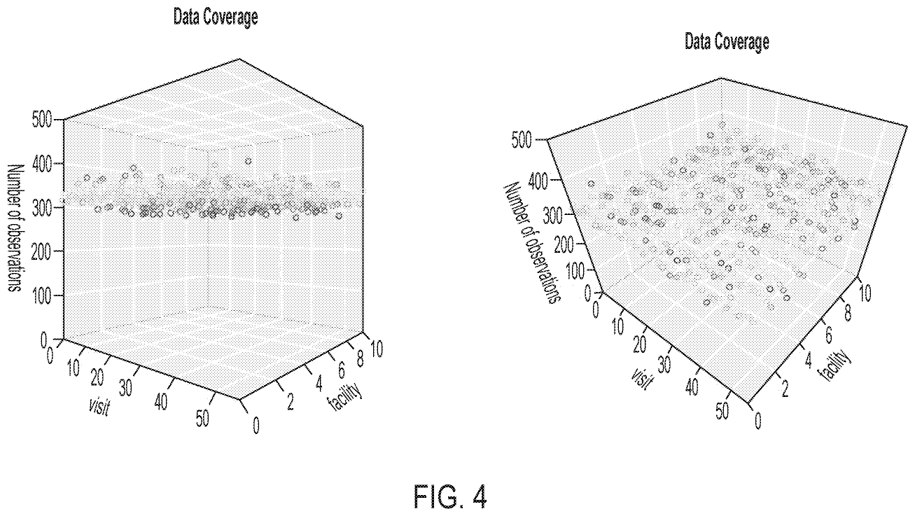

FIG. 4 shows two graphs of the number of observations in the above-mentioned data. The graphs show the distribution of observations across the facility decile and the number of visits to the facility.

### Prediction and Decision Models

The company used a random forest model as the predictive model, with the target variable Y being the deviation in sales to a facility from the mean sales of facilities in the same decile of sales as the facility. Random forest models are ensemble machine learning models that can perform both regression and classification. Random forest models may merge predictions from multiple decision trees to achieve a more accurate and stable prediction than a single decision tree. Each decision tree in a random forest may learn from a random sample of training data. By training each tree on different samples, the random forest model may achieve low variance.

The above-mentioned features explained 72% of the variation in sales. The importance of each feature is shown in Table 1 below.

TABLE 1
Random Forest Variable Importance

| | % IncMSE | IncNodePurity |
|---|---|---|
| qtr | 133.16188 | 4.089630e+13 |
| visit | 125.76667 | 1.780477e+14 |
| facility | 102.33283 | 7.961670e+13 |
| email | 84.30202 | 8.880591e+13 |
| appointment | 77.56009 | 1.281616e+14 |
| group | 56.38927 | 5.964828e+13 |
| conference | 47.18138 | 3.337538e+13 |
| product | 31.50224 | 7.460655e+12 |

Table 1 shows that quarter, visit, and facility are the most important predictors. % IncMSE, for a particular variable, measures how much the model would degrade in predictive ability if data from the variable were to be replaced with random noise. IncNodePurity measures a degree to which data in nodes split by particular variables are homogeneous. Splitting the tree into nodes that are more homogeneous may result in improvement in the predictive and ranking power of the model and hence in improvements in the quality of the decisions made based on the model.

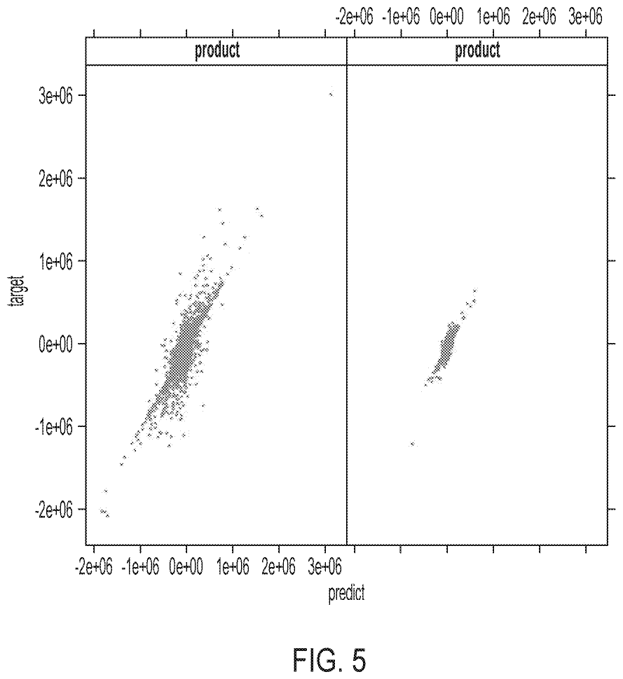

FIG. 5 is a scatter plot of the predictive model's predicted values against the actual target values for each of the therapeutics. The plots show a strong diagonal pattern which confirms that the model fit is good. As described above, the approaches to building an explanation model evaluate the predictive model either on a sample of the data set used to train the model or on a set of counterfactuals. In this case, counterfactuals were used to generate observations that cover the complete space of the predictors. The system may use these data to build the decision model.

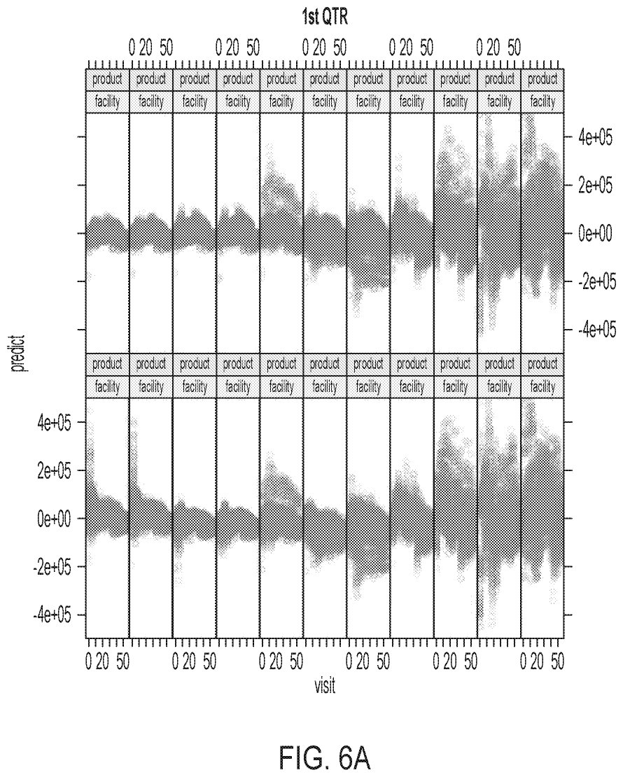

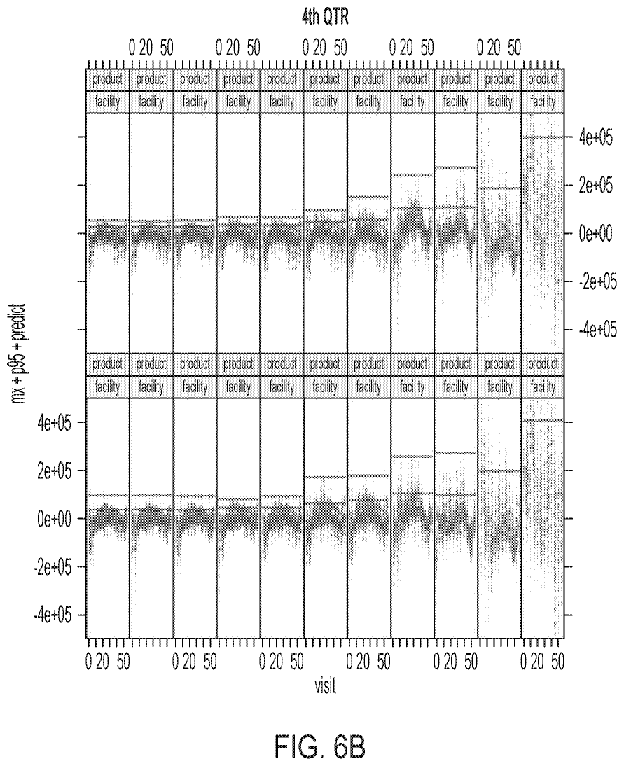

The surface defined by the predictions of f( ) is an 8-dimensional surface. Since the observations that comprise the surface are from the predictions of a random forest model and not a parametric model, there are discontinuities in the surface, as the plots of FIGS. 6A and 6B show. FIGS. 6A and 6B show the surface across 4-dimensions for two quarters. The surface varies across the quarters, across the facilities, and across the products. The first row in each plot shows data for product 1 and the second is for product 2; as the plots move from left to right the facility decile increases. Some of the variance and fluctuations in the plots is caused by the discontinuities of the random forest model and some is caused by the hidden variables that are not shown in the plots. FIGS. 6A and 6B illustrate more detail on this prediction surface and provides insight into the decision model. The plot on the right has blue and red lines—these are the maximum and the 95% quantile for the prediction in each of the identified dimensions, respectively. The value of visit where the maximum intersects is the value for d*(x) for that set of predictors. Since there is variance associated with the predictive model method, the average number of visits where the prediction for those values is above the 95% quantile within that bin of predictors is used as the value for d*(x).

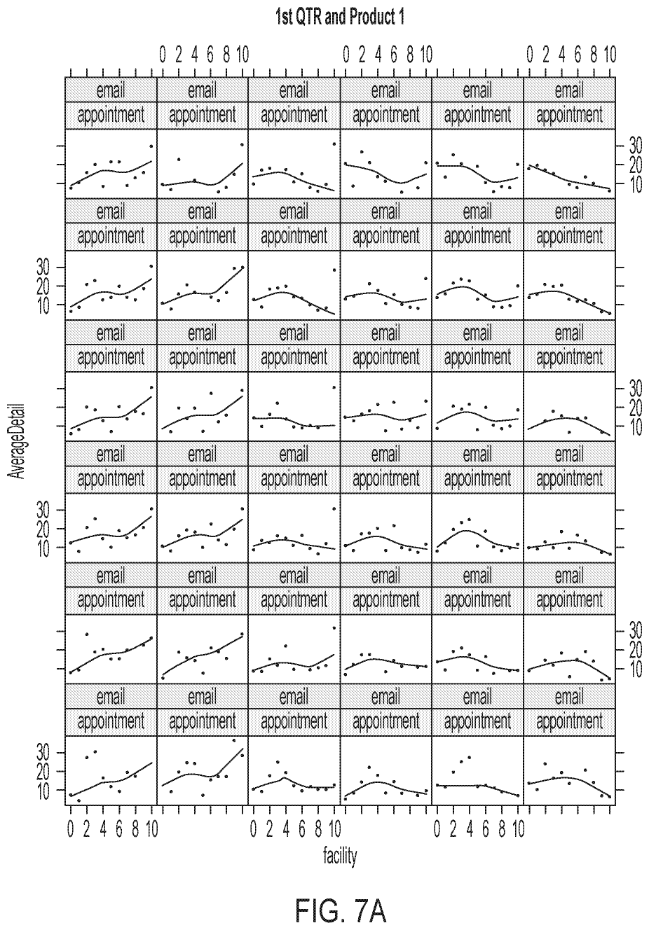

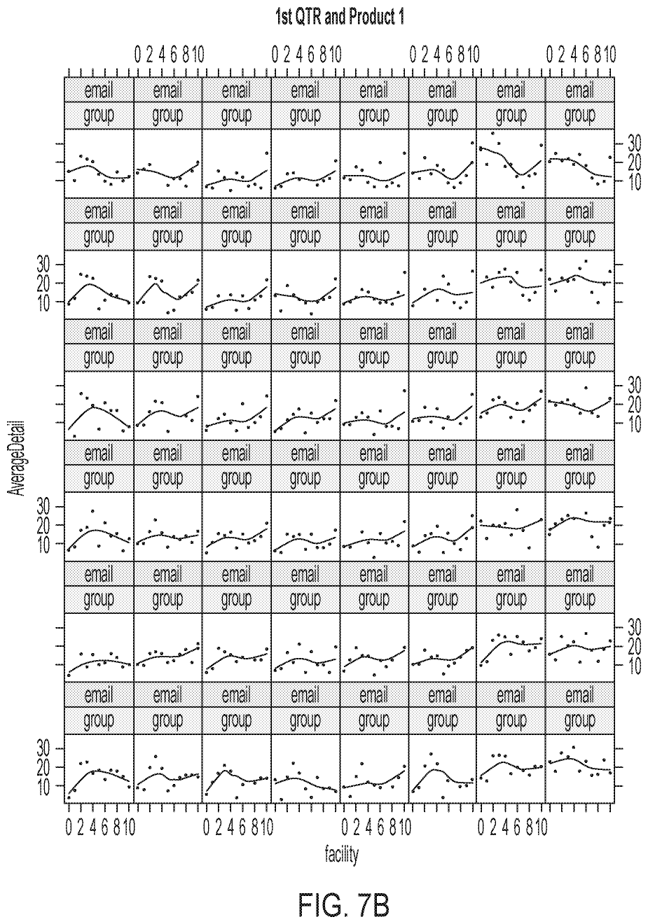

FIGS. 7A and 7B include plots that depict the average number of visits above the 95% quantile for several combinations of predictors. They also show a smoothed kernel estimated line through those points.

In the plots on the left, the estimate lines show the number of visits that maximize sales as a function of facility sales size, number of emails sent, and number of appointments. Appointments increase for the plots further to the right and emails sent increase for the plots further toward the top. The plots show that the value of visits increases with facility size when there are fewer appointments (estimate lines with positive slopes on the left), but that trend inverts as appointments grow (kernel estimator lines with negative slopes on the right). One might expect that appointments are more important as facilities grow. The plots on the left also show that the impact of the number of emails sent is more subtle (only a small variation in the slope of the estimate lines in the same column).

The plots on the right are similar but focus on the number of group meetings in place of appointments. Group meetings increase in the plots to the right and emails sent increase in the plots towards the top of the page. Group meetings may be more cost effective since a number of prescribers are simultaneously in a meeting. The data suggest that more visits are needed as facility size grows. This may suggest that the HCPs need more explanation in face-to-face visits after group meetings. Since these are views of marginal slices through the decision space it is difficult to get a complete understanding of the drivers and shape of d*( ). As such, an explainability model is desirable.

### Explanation Models

Marginal plots, across some dimensions of input data, may provide insight into an underlying decision model, but it may not capture all interactions and their relative strengths. Further, using linear models such as LIME and CLEAR may not give full insight into the relative impacts of all of the variables on the optimal decision produced by the decision model. For a particular decision point, it may be useful to determine what factors make the particular decision point optimal or desired, as well as how particular values of decision variables result in this particular decision point. To capture more interactions, the system may test multiple solutions close to an optimal solution and recurse to determine values of decision variables associated with the multiple solutions.

As a first step to gaining a deeper explanation, the company may fit a decision tree to d*(x) using recursive partitioning. Recursive partitioning is a statistical method for multivariable analysis. Recursive partitioning creates a decision tree that strives to correctly classify members of the population by splitting it into sub-populations based on several dichotomous independent variables. The recursive partitioning enables insight into covariate relationships in d*(x).

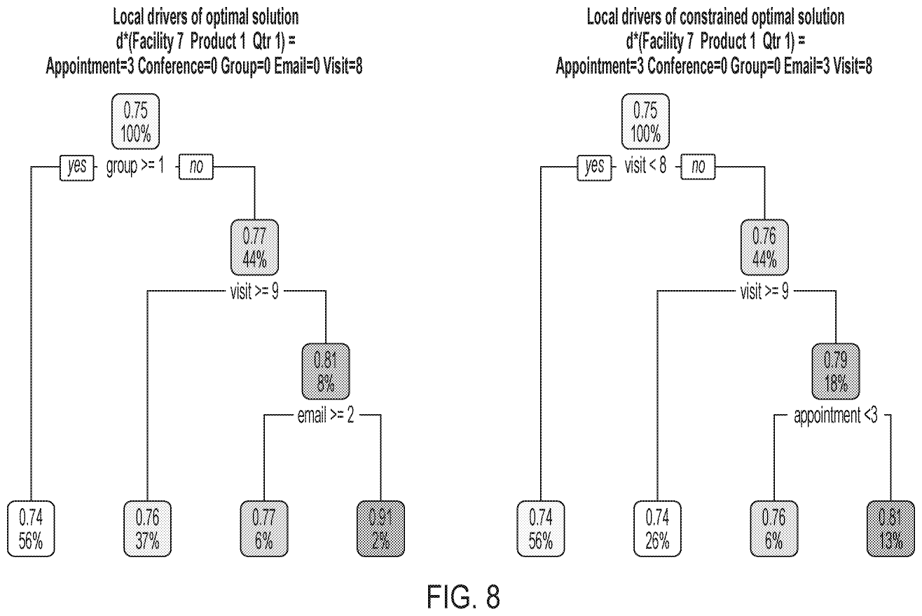

FIG. 8 shows two trees fit to predict proximity to an instance d*(z) (where z is a transformation of x) by using all solutions within 70% of an optimal solution (e.g., maximum sales) as a target. The left tree shows results for an unconstrained decision model, while the tree on the right shows results for a constrained model. For the tree on the left, the top node labeled 0.75 and 100% indicates that the solutions for decision variables in all sub-nodes of the tree average 75% of the optimal solution for d*(Facility 7 Product 1 Qtr 1). The subgroup of this tree having group value 1 represents 56% of the population and has a mean percent of optimal of 74%. The tree also shows that the optimal solution may have visits of 8 or less, have 0 or 1 emails sent to HCPs within Facility 7, and achieve 91% optimal sales. The decision variables not included in the tree may not be drivers of the optimal solution. The tree may be considered a local explanation that gives insight into variables that impact optimality within a neighborhood of the solution d*(z*).

The constrained decision model may incorporate one or more constraints. For example, the constrained model may incorporate a constraint requiring a number of emails sent to be at least 25% of the number of visits. The tree to the right shows that the “visit” variable is the most consequential in driving to the optimal solution. The decreased value for the optimum solution shown in this plot may reflect the constraint in place.

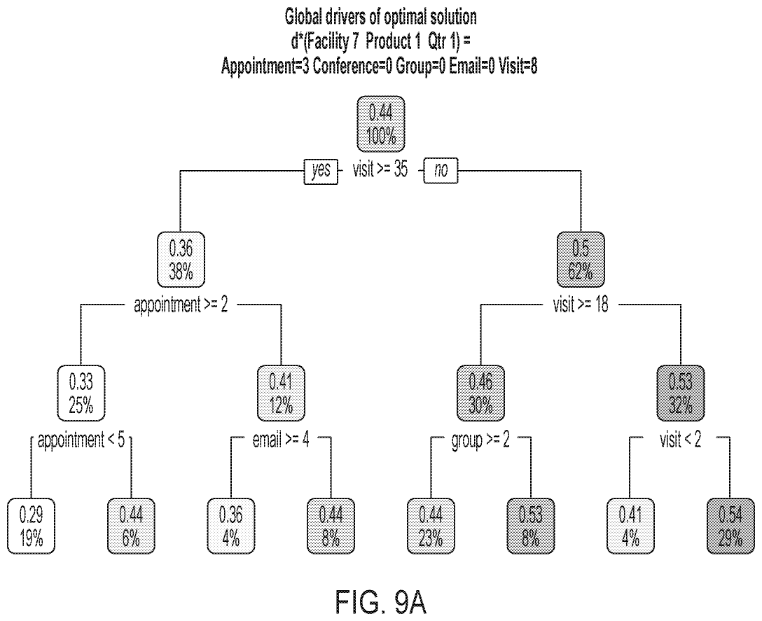

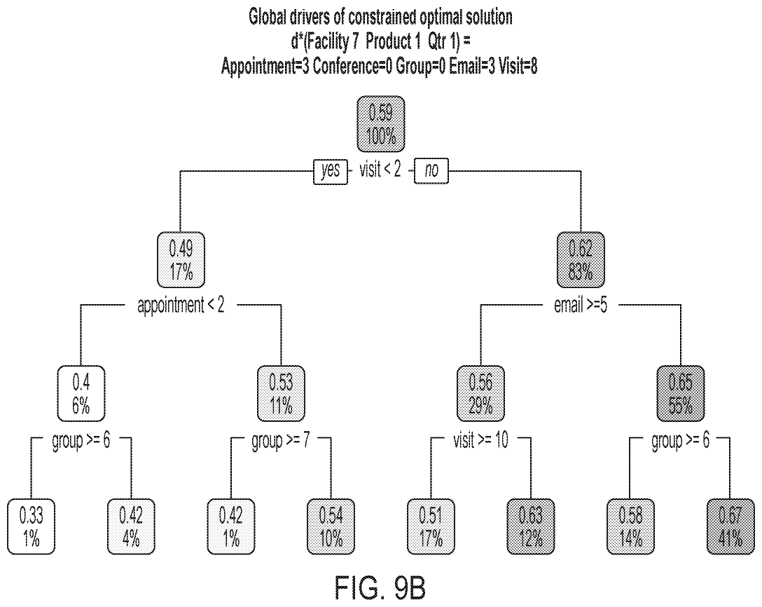

FIGS. 9A and 9B show decision trees for a global explanation model. Instead of restricting the search space to within 70% of the optimum, the entire space of predictions on all the counterfactuals may be used in the recursive partitioning algorithm. In the example unconstrained and constrained trees of FIGS. 9A and 9B, the order of splits in the trees tracks with the order of importance—the variables towards the top of the tree are more important for producing the optimal solution. The constrained analysis in the tree on the right shows the impact of the constraint driving emails into the solution. The branch on the right labeled “email>=5” contains 83% of the space and accounts for a mean 62% of the constrained optimum value. The sub-branches show there is a tradeoff between “group” visits and “visits” that help the decision model navigate the email constraint.

### Local Explanation Model

This example has focused on a global approach to explaining d*(x). However, recursive partitioning can be used to obtain an explanation on a more localized portion of the problem. In explainability approaches like LIME and CLEAR, local explainability is obtained by analyzing the behavior of the underlying model at a single point by sampling the underlying model in the space around that point. In the case of LIME, a linear model is built based on those points as described previously in this disclosure. In the previous section, recursive partitioning was used on the entire space of d*(x). The example of FIGS. 9A and 9B focus on a portion of the space. Since facility size is an important predictor and an important variable for the decision model, recursive partitioning can be applied to a single value of facility.

FIGS. 9A and 9B show a decision tree for different sales deciles (the third and eighth deciles) of facilities. Both analyses were done to the same split level. While quarter is the most important variable for the first split in each case, the structure below that differs significantly. This is expected since this analysis is conditioned on the three most important variables identified in Table 1. These trees show the relationship between the number of visits that maximize sales given the number of appointments, the number of group meetings, and the number of conferences for the margins defined by quarter, product, and facility size.

### LIME Explanation Model

An implementation of the LIME algorithm was developed for this example. The standard implementation samples from the test set and then builds a linear model using predictions on the sample points weighted by distance to the point of interest. The coefficients associated with the linear explanation model yield the importance of the predictors for that particular explanation point. The current implementation has been modified to use counterfactuals across the entire space used to evaluate d*(x). Similar to the standard LIME approach, a point is sampled, but then rather than sampling additional points around the point of interest, all counterfactuals within a hypercube with side length 1 are sampled. This example has all integral predictors, so a unit hypercube is a natural choice. If some of the predictors were continuous, a similar approach could be taken, although a different strategy would be needed to evaluate the decision model counterfactuals.

The current implementation also weights the observations in the LIME explanation model by exp(−w), where w is the distance from the points in the hypercube to the point of interest.

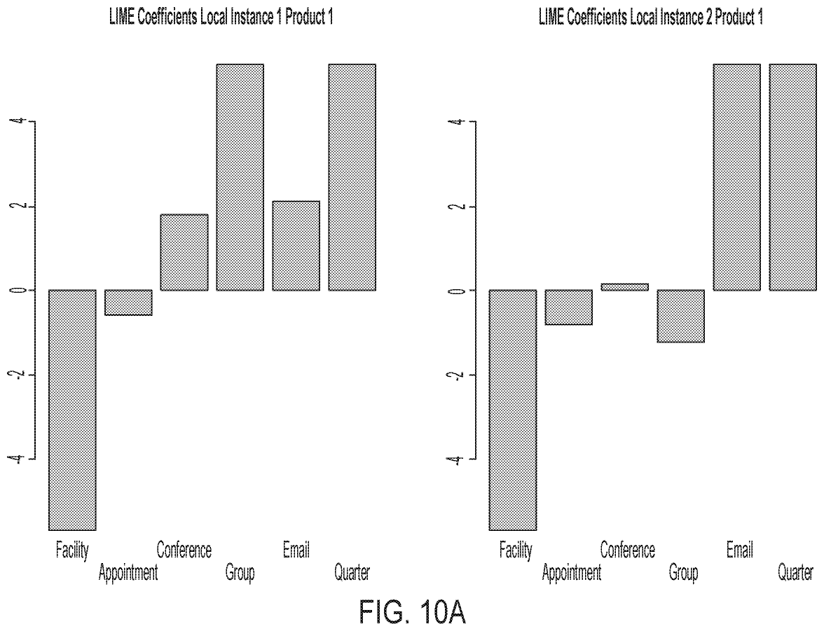

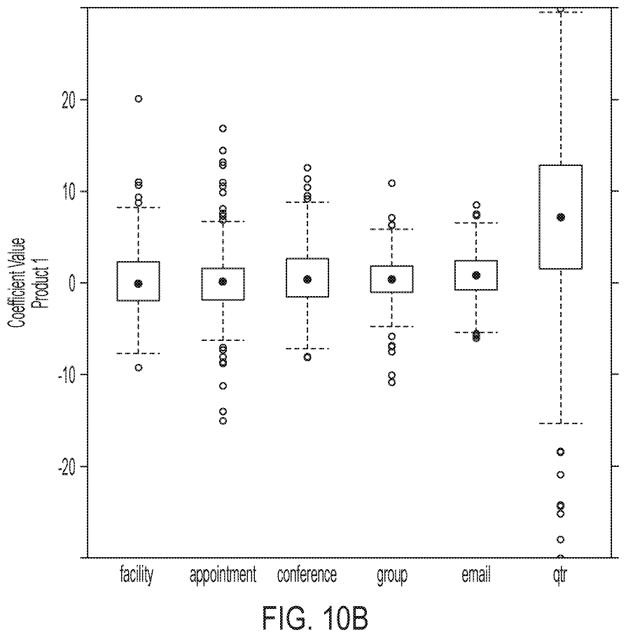

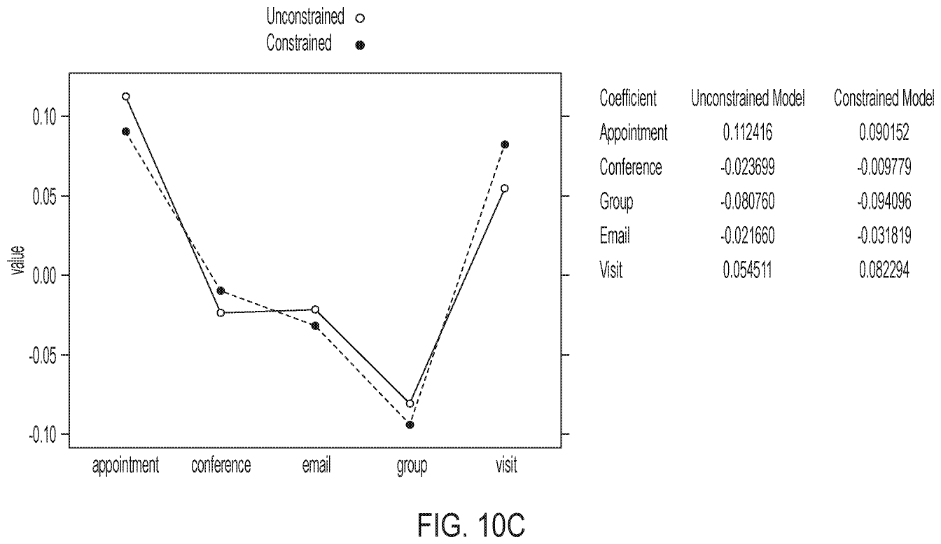

The bar charts in FIG. 10A show the coefficient values for the sampled instances. Positive values are interpreted as increases in the predictor driving increases in the number of visits that optimize sales. Notice that in the observations, increasing the quarter is associated with increasing visits in the sales-optimizing scenarios. This is consistent with the observations from the recursive partitioning explanation models as shown, for example, in FIGS. 8 and 9A and 9B.

While LIME is a local explanation approach, it can be used to understand how a model behaves more generally by examining the explanation coefficient across a large number of sample points of interest. For example, one may pick a set of instances for the user to inspect and then display the result in a matrix of instances. Here, we sample a small number (250) of points of interest and show box plots of the coefficient values in FIG. 10B. The plot shows the strong influence of the quarter on the optimal number of visits for maximizing sales. What cannot be seen in this plot is the detail that the recursive partitioning reveals in, for example, FIGS. 9A and 9B, where for smaller facilities it is favorable to have fewer appointments in the second half of the year in comparison to larger facilities, where it is favorable to have more appointments in the first half of the year.

The system may build a linear model with the weighted hypercube values as predictors, with the target of the model a percent deviation from an optimal value. This may be the same target as used in recursive partitioning.

The system may build a linear model with the weighted hypercube values as predictors. To determine contributions of predictor variables (predictor), the system may test different model targets that are particular percentage deviations from an optimum value. The plot of FIG. 10C shows values of coefficients for two estimated LIME explanation models: for a constrained model and for an unconstrained model. The predictors are the horizontal axis and the values of their coefficients are on the vertical access. The table gives the exact values of the coefficients. The value of r2 for the unconstrained model is 0.97 and for the constrained model is 0.98, meaning that the model is an effective predictive tool. The plot shows that, for each model, the variables “appointment”, “conference”, and “visit” were highly determinative of the prediction. Although these results match those for the recursive model, the LIME model may not determine the multivariate impact of the predictors as explainers in the decision model.

### Computer Systems

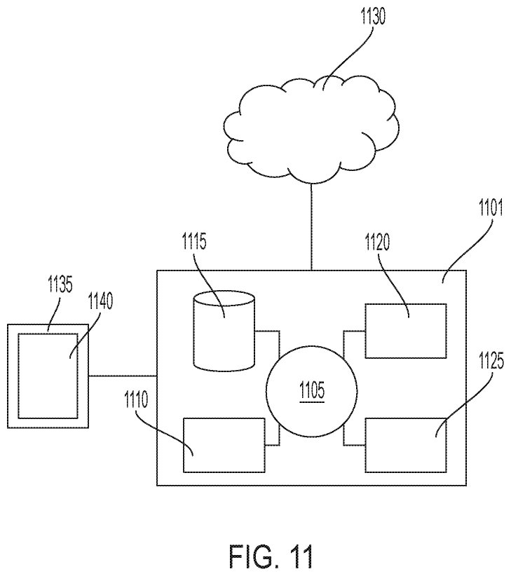

The present disclosure provides computer systems that are programmed to implement methods of the disclosure. FIG. 11 shows a computer system 1101 that is programmed or otherwise configured to implement the predictive models, decision models, and explanation models described herein. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user (e.g., the user's mobile device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine-readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example, visualizations of explanation models such as decision trees. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, be a predictive model or decision model.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for providing information about the structure and function of an optimization machine learning model, comprising:
(a) generating a predictive machine learning model, wherein the predictive machine learning model predicts a value for a target variable from a set of features;
(b) generating a decision machine learning model from the predictive machine learning model, wherein the decision learning model predicts one or more values for one or more decision variables, wherein a decision variable is a feature from the set of features, wherein the decision machine learning model determines one or more decision variables which provide a maximum value of the target variable;
(c) generating a plurality of target variable predictions using the predictive machine learning model, wherein each of the plurality of target variable predictions approximates the maximum; and
(d) determining, using a recursive machine learning algorithm, values of decision variables associated with the plurality of target variable predictions.

2. The method of claim 1, wherein the recursive machine learning algorithm is a decision tree algorithm.

3. The method of claim 2, wherein the determining the values of the decision variables is performed via recursive partitioning.

4. The method of claim 3, wherein the recursive partitioning uses the entire space of the decision model.

5. The method of claim 3, wherein the recursive partitioning uses a portion of the space of the decision model.

6. The method of claim 1, wherein the predictive model is a binary classifier.

7. The method of claim 1, wherein the decision machine learning model is constrained by restricting a searchable space of decision variables.

8. The method of claim 7, wherein restricting the searchable space comprises applying one or more rules.

9. The method of claim 1, wherein generating the plurality of target variable predictions comprises perturbing one or more features of the set of features.

10. The method of claim 1, wherein the set of features comprises demographic data from human subjects.

11. The method of claim 1, wherein a decision variable is associated with control by a human subject.

12. The method of claim 11, wherein the decision variable is an action.

13. The method of claim 1, wherein the predictive model comprises a neural network.

14. The method of claim 13, wherein the neural network is a convolutional neural network (CNN) or a recurrent neural network (RNN).

15. The method of claim 1, wherein the predictive model comprises a decision tree.

16. The method of claim 15, wherein the predictive model comprises a random forest.

17. The method of claim 1, wherein each of the plurality of target variables falls within 70% of the maximum.

18. The method of claim 1, wherein the target variable is a discrete variable, a continuous variable, or a binary variable.

19. The method of claim 1, wherein the recursive machine learning algorithm iterates until a stopping criterion is reached.

20. A system for providing information about the structure and function of an optimization machine learning model, comprising:
a computer database configured to contain encrypted health data; and
one or more computer processors individually or collectively programmed to:
(a) generate a predictive machine learning model, wherein the predictive machine learning model predicts a value for a target variable from a set of features;
(b) generate a decision machine learning model from the predictive machine learning model, wherein the decision learning model predicts one or more values for one or more decision variables, wherein a decision variable is a feature from the set of features, wherein the decision machine learning model determines one or more decision variables which provide a maximum value of the target variable;
(c) generate a plurality of target variable predictions using the predictive machine learning model, wherein each of the plurality of target variable predictions approximates the maximum; and
(d) determine, using a recursive machine learning algorithm, values of decision variables associated with the plurality of target variable predictions.
